(19) United States
(12) Patent Application Publication
(10) Pub. No.: US 2024/0325667 A1
(43) Pub. Date: Oct. 3, 2024

(54) ENDOTRACHEAL TUBES WITH BAFFLES

(71) Applicants: University of Pittsburgh - Of the Commonwealth System of Higher Education, Pittsburgh, PA (US); UPMC, Pittsburgh, PA (US)

(72) Inventors: Carl H. Snyderman, Pittsburgh, PA (US); Garrett N. Coyan, Pittsburgh, PA (US)

(73) Assignees: University of Pittsburgh - Of the Commonwealth System of Higher Education, Pittsburgh, PA (US); UPMC, Pittsburgh, PA (US)

(21) Appl. No.: 18/579,199

(22) PCT Filed: Jul. 14, 2022
(86) PCT No.: PCT/US2022/037087

§ 371 Date: Jan. 12, 2024

Related U.S. Application Data
(60) Provisional application No. 63/221,572, filed on Jul. 14, 2021.

(51) Int. Cl. A61M 16/04; A61M 16/00

(52) U.S. Cl. CPC A61M 16/0431; A61M 16/0003; A61M 16/0465; A61M 2016/0027; A61M 2205/0216; A61M 2205/3327; A61M 2205/3331; A61M 2210/1032

(57) ABSTRACT

Disclosed herein are endotracheal tubes that have external baffles near the distal end of the tube to reduce unwanted migration of the tube relative to the trachea and to help prevent materials from passing between the outside of the tube and the tracheal walls. The baffles can be made of soft flexible material that conforms to and seals against the trachea. The baffles can be angled proximally or distally, or both. The baffles can be tapered in thickness and can vary in radial length. Some embodiments can include a supraglottic umbrella. Some embodiments can include an axially expandable portion along the tube. Some embodiments can include a loading tube that helps set the deflection orientation of the baffles within the trachea.

### CROSS-REFERENCE TO RELATED APPLICATIONS

This application claims the benefit of U.S. Provisional Patent Application No. 63/221,572 filed Jul. 14, 2021, which is incorporated by reference herein.

### FIELD

This application relates to endotracheal tubes and similar tubes for placement within other hollow organs or cavities in the body.

### BACKGROUND

Conventional endotracheal tubes include an inflatable cuff to provide a seal for ventilation. Problems with such tubes include unwanted aspiration around the cuff resulting in ventilator-associated pneumonia, pressure necrosis from over-inflation of the cuff, unwanted tube migration (proximally and distally) with risk of hypoventilation or accidental extubation, and cuff malfunction (e.g., leakage). Ventilator associated pneumonia is associated with significant morbidity and mortality and increases the cost of care by very significant amounts per episode.

### SUMMARY

Disclosed herein are endotracheal tubes that have external baffles near the distal end of the tube (e.g., in lieu of inflatable cuffs) to reduce unwanted migration of the tube relative to the trachea and to help prevent aspiration of materials between the outside of the tube and the tracheal walls. The baffles can be made of soft, flexible material that is resiliently deformable and that conforms to and seals against the tracheal walls. In some embodiments, the device can include two or more sets of baffles, such as with each set including differently shaped or oriented baffles. The baffles can be angled proximally or distally, or both, to provide biased properties in one direction of the other. The baffles can be tapered in thickness and can vary in radial length. Some baffles can be helically coiled around the tube. Some embodiments can include a supraglottic umbrella to further help prevent aspiration. Some embodiments can include an axially expandable portion along the tube to adjust the length between device features. Some embodiments can include a loading tube that helps set the deflection orientation of the baffles within the trachea and then is removed before the device is used. Some embodiments can include a plurality of flexible protuberances extending from the outside of the tube, either instead of or in addition to baffles. Some embodiments can include suction channels to remove materials from around the baffles. Some embodiments can include a forked distal end portion with each fork having its own baffles. Some embodiments can have baffles formed separately from the tubular portion, such as from different materials.

The foregoing and other features, variations, and advantages of the disclosed technology will become more apparent from the following detailed description, which proceeds with reference to the accompanying figures.

### BRIEF DESCRIPTION OF THE DRAWINGS

FIG. 1 shows a partial cross-section of a patient's airway with an exemplary endotracheal tube with baffles inserted into the trachea.

FIG. 2 shows the exemplary endotracheal tube with baffles in isolation.

FIG. 3 shows an embodiment having perpendicular baffles.

FIG. 4 shows an embodiment having distally angled baffles.

FIG. 5 shows an embodiment having proximally angled baffles.

FIG. 6 shows an embodiment having a helical baffle.

FIG. 7 is a cross-sectional view of an embodiment having tapered baffles.

FIG. 8 is a cross-sectional view of an embodiment having baffles that vary in diameter.

FIG. 9 is a cross-sectional view of an embodiment having baffles that each include both a proximally angled portion and a distally angled portion.

FIG. 10 is a cross-sectional view of an embodiment having a helical baffle of varying diameter.

FIG. 11 is a cross-sectional view of an embodiment having angled baffles.

FIG. 12 is a cross-sectional view of an embodiment having one set of distally angled baffles and one set of proximally angled baffles, with the two sets tilted toward each other.

FIG. 13 shows an exemplary endotracheal tube having one set of distally angled baffles and one set of proximally angled baffles, with the two sets tilted away from each other.

FIGS. 14-16 show views of another embodiment having one set of distally angled baffles and one set of proximally angled baffles, with the two sets tilted away from each other.

FIG. 17A shows an exemplary endotracheal tube having two sets of proximally angled baffles.

FIG. 17B shows the tube of FIG. 17A being inserted distally into a trachea, causing the baffles to defect proximally.

FIG. 17C shows the tube of FIG. 17B being retracted proximally, causing the baffles to defect distally.

FIG. 18A shows an exemplary endotracheal tube having a distal set of distally angled baffles and a proximal set of proximally angled baffles, and a loading tube being passed over the baffles.

FIG. 18B shows the baffles deflecting proximally when the loading tube is moved proximally over the baffles.

FIG. 18C shows the loading tube being moved proximally after the endotracheal tube with loading tube covering the baffles is inserted into the trachea. In FIG. 18C, the loading tube uncovers only the distal baffles. In this position, the endotracheal tube can be moved proximally a small distance to cause the distal baffles to deflect distally against the tracheal wall.

FIG. 18D shows the loading tube being moved further proximally to uncover the proximal baffles as well, which are deflected proximally against the tracheal wall.

FIG. 19A shows an exemplary endotracheal tube having perpendicular baffles and a supraglottic umbrella.

FIG. 19B shows the tube of FIG. 19A inserted into the trachea with the supraglottic umbrella positioned near the proximal end of the trachea, where the glottis would be.

FIG. 20 shows an exemplary endotracheal tube having two spaced apart sets of baffles and a supraglottic umbrella.

FIG. 21 shows an exemplary endotracheal tube having a set of perpendicular baffles, a supraglottic umbrella, and axially expandable baffles therebetween.

FIG. 22 shows an exemplary endotracheal tube having flexible protuberances along the outer surface of the tube.

FIG. 23 is a cross-sectional view of the tube of FIG. 22.

FIG. 24 shows another exemplary endotracheal tube having flexible protuberances along the outer surface of the tube.

FIG. 25 is a cross-sectional view of the tube of FIG. 24.

FIG. 26 shows an exemplary endotracheal tube having a set of baffles with curled edges.

FIG. 27 shows an exemplary endotracheal tube having a set of broad baffles to seal the airway, a smaller baffle near a distal tip to prevent contact with the tracheal wall, and a proximal baffle at the subglottic region.

FIG. 28 shows an endotracheal tube having a supraglottic umbrella positioned over the glottis.

FIG. 29 is a cross-sectional side view of the endotracheal tube of FIG. 28.

FIG. 30 shows a non-circular cross-sectional profile for an endotracheal tube, with the posterior aspect having a flattened wall.

FIG. 31 shows a generally triangular cross-sectional profile for an endotracheal tube.

FIG. 32 is a cross-sectional view of an endotracheal tube having a non-circular baffle that has a flat posterior aspect.

FIG. 33 shows an endotracheal tube that includes a helical sensor embedded in the wall of the tube.

FIG. 34 shows an endotracheal tube that includes a set of baffles and sensors embedded in the baffles.

FIG. 35 shows an endotracheal tube that includes a series of axially spaced apart sensors along the tube.

### DETAILED DESCRIPTION

Disclosed herein are endotracheal tubes that comprise external circumferential baffles near a distal end of the tube, which provide a seal for ventilation, reduce unwanted motion of the tube relative to the trachea, and restrict materials from passing between the outside of the tube and the tracheal walls (e.g., aspiration). The baffles can replace and improve the functionality of a conventional inflatable cuff at the distal end of the tube. In some embodiment, baffles can be included in addition to one or more inflatable cuffs. As described herein, the disclosed baffles can have many different shapes, sizes, placements, orientations, groupings, and numbers. The baffles can comprise a series of soft, flexible, annular membranes that extend outwardly from the outer surface of the tube and provide circumferential contact with the tracheal mucosa, helping to prevent aspiration and migration while sealing the airway for ventilation.

Any of the devices, methods, and other features disclosed herein can be combined in any combination, and can be combined with any of the devices methods, and other features (such as one or more inflatable cuffs) disclosed in U.S. Provisional Patent Application No. 63/221,547 filed on Jul. 14, 2021, entitled “MULTI-CUFFED ENDOTRACHEAL TUBES,” and the co-pending International PCT Application filed on the same day as this application, also entitled “MULTI-CUFFED ENDOTRACHEAL TUBES,” which are incorporated by reference herein in their entirety.

In some embodiments, additional features can be included, such as a supraglottic umbrella proximal to the baffles and a loading tube to help orient the baffles properly when inserted into the trachea.

The embodiments disclosed herein can include any one or more of the following features and/or other features:
1. Multiple circumferential baffles that line the exterior surface of the endotracheal tube. The baffles are soft and pliable so that they do not hinder tube insertion.
2. Multiple baffles provide adequate seal of airway for positive pressure ventilation.
3. Pliable baffles allow minimal escape of air with positive pressure ventilation which prevents pooling of secretions and aspiration.
4. Angulation of baffles resists movement of the tube.
5. Multiple groups of baffles with different angulation resists distal and proximal movement of the tube.
6. Varied lengths of groups of baffles allows “anchoring” of tube.
7. Proximal or distal suction channels.
8. Pressure sensors on baffles that detect movement of tube, orientation of baffles, and/or degree of bending/flattening of baffles.
9. Proximal umbrella that secures the tube above the supraglottis.
10. Proximal umbrella that prevents aspiration of gastric contents and directs pharyngeal contents toward the esophagus.
11. Translucent materials that improve visualization.
12. Sterilizable and re-usable.

The disclosed embodiments can provide any one or more of the following advantages over conventional endotracheal tubes and/or other advantages:
1. Absence of an inflatable cuff. This avoids cuff-related morbidity and malfunction.
2. More effective at preventing aspiration, the major cause of ventilator associated pneumonia.
3. Decreases the risks of tube migration with associated morbidity.
4. Easier to use with minimal learning curve.
5. Improved adoption of use.
6. Decreased production costs compared to expensive modified tubes and monitoring devices.
7. Decreased monitoring of cuff pressures and tube position by healthcare personnel.
8. Suitable for pediatric patients.
9. Sterilizable and re-usable for resource-poor environments.
10. Resists movement from high ventilation pressures.
11. Allows escape of high ventilation pressures.

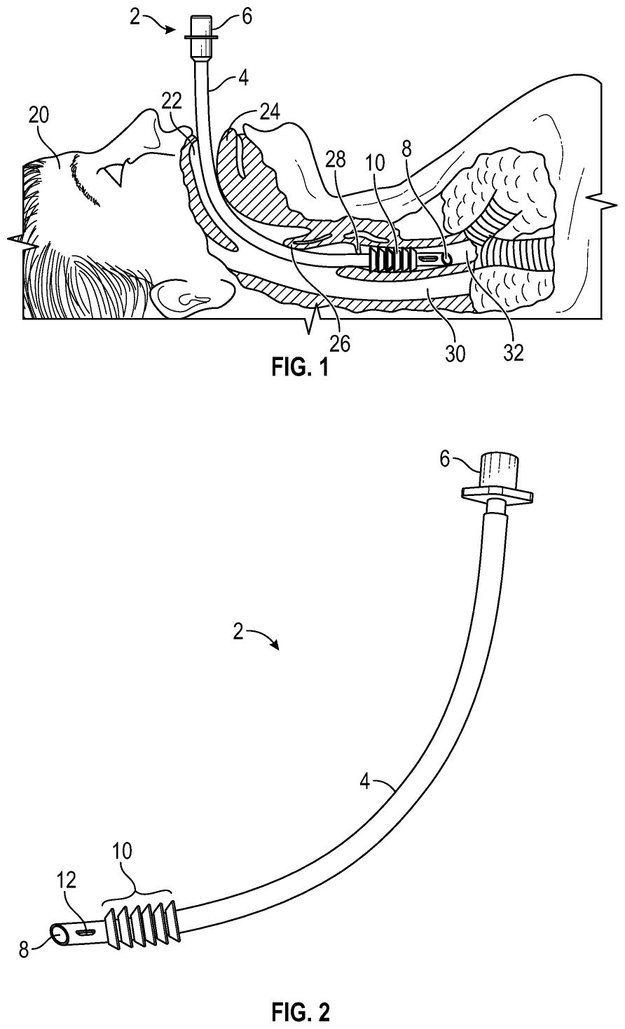

FIG. 1 shows a partial cross-section of a patient 20 illustrating the airway with an exemplary endotracheal tube with baffles 2 inserted into the trachea. FIG. 2 shows the endotracheal tube (also referred to as the “device”) 2 in isolation. The device 2 can comprise a tubular portion 4, an inlet adaptor 6 at a proximal end of the tubular portion for coupling to a ventilator or other device, a distal outlet 8, a lateral outlet 12, and annular baffles 10 positioned around a distal portion of the tubular portion 4. As shown in FIG. 1, the device 2 is inserted through the mouth 22, over the tongue 24, past the epiglottis 26, through the larynx 28, and into the trachea 32. The esophagus 30 is also shown. The baffles 10 are soft and flexible and conform to the shape of the trachea at a location between the larynx and the bronchi. The baffles 10 gently engage the walls of the trachea 32 to hold the device 2 in the desired position, such as with the distal outlet 8 proximal to the bronchi. In some embodiments, the device prevents distal migration into one bronchus and avoids obstruction of the other bronchus, thereby maintaining ventilation of both lungs. The baffles 10 create a seal to support ventilation while blocking aspiration between the device 2 and the trachea 32 and resisting unwanted axial movement of the device.

At the same time, the baffles can be soft and flexible enough to allow proximal release of air past the baffles during high positive pressure in the lungs during ventilation. The softness of the baffles and the distribution of the baffles along the tube can also help more evenly distribute pressure across a larger area and reduce the risk of pressure necrosis of the tracheal mucosa.

In any of the embodiments described herein, the baffles can include pressure sensors to provide pressure data to a monitoring device, e.g. via wires running along the tube and/or via wireless connections. Such sensors can be used to detect excess pressure or insufficient pressure, and to detect migration of the device.

In any of the embodiments described herein, the device can include integrated suction channels, conduits, or catheters running along the tubular portion and coupled to openings in the walls between or adjacent to the baffles. Such suction conduits can be used to suck fluids or other materials that build up between or adjacent to the baffles. For example, suction openings can be positioned between each adjacent pair of baffles, just above the most proximal baffle of each set of baffles, and/or just below the most distal baffle of each set of baffles. Removing excess materials around the baffles can help prevent unwanted aspiration and infections.

In any of the embodiments described herein, the tubular portion of the device can include reinforcing members, such as wires, bars, coils, rings, etc, to help prevent kinking, compression, or other unwanted deformation of the tubular portion. In some embodiments, the baffles can also include reinforcing members.

In some embodiments, the tubular portion can comprise a double lumen that forks into two distal end portions, or forks, for independent ventilation of each lung. Each fork can terminate in or adjacent to a respective one of the bronchi. In such embodiments, each fork can have its own set of baffles. In some embodiments, a larger, proximal set of baffles is located around the tube before the fork, and one or more smaller sets of baffles are located around each fork.

In some embodiments, the tubular portion and the baffles can be co-formed (e.g., co-molded) of a common material. In other embodiments, the tubular portion and the baffles can be separately formed and later coupled together. For example, the baffles and the tubular portion can be formed of different materials that have different properties. In some embodiments, the tubular portion and/or the baffles can comprise composite construction, being constructed of a combination of two or more different material. Such material can comprise various polymeric materials, metallic/alloy materials, reinforcing components, outer coatings, radiographic elements, visual markings, etc.

In some embodiments, the tubular portion and the baffles can be made of a single-molded material with variable thickness and therefore variable rigidity and flexibility.

In some embodiments, the baffles and/or outer surface of the tubular portion can be coated or impregnated with various substances, such as an antimicrobial agent, non-stick material, etc.

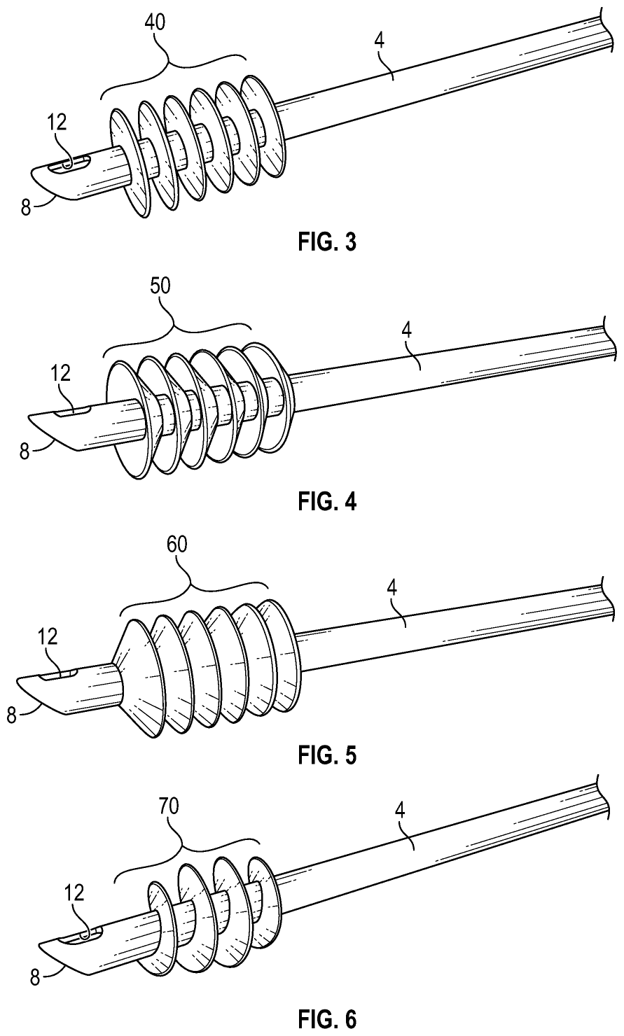

FIGS. 3-12 show various different types of exemplary baffles, any of which can be used in a manner similar to FIG. 1. FIG. 3 shows an embodiment having thin, disk-shaped baffles 40 that extend perpendicularly from the tubular portion 4. FIG. 4 shows an embodiment having distally angled baffles 50 that have a frustoconical shape. The distally angled baffles 50 can be more resistant to unwanted distal migration, while allowing the device 2 to be moved proximally (e.g., removed) with less resistance. FIG. 5 shows an embodiment having proximally angled baffles 60 (similar to baffles 10) that also have a frustoconical shape but facing the opposite direction. The proximally angled baffles 60 can be more resistant to unwanted proximal migration (e.g., accidental extubation), while allowing the device 2 to be moved distally (e.g., insertion) with less resistance. FIG. 6 shows an embodiment having a helical baffle 70 that coils around the tubular portion 4. The helical baffle 70 can also vary in diameter, such as having a smaller diameter at the ends and larger diameter in the middle. A helical baffle can have the unique property of a continuous helical pathway between the turns of the baffle that can allow secretions and other materials to pass through the baffle without stasis, which can help avoid unwanted build-up around the baffles. The helical baffle can also help avoid focal circumferential pressure along an annular ring section of the trachea. Helical baffles can also provide enhances resistance to torsional/rotational motion of the tube with the trachea.

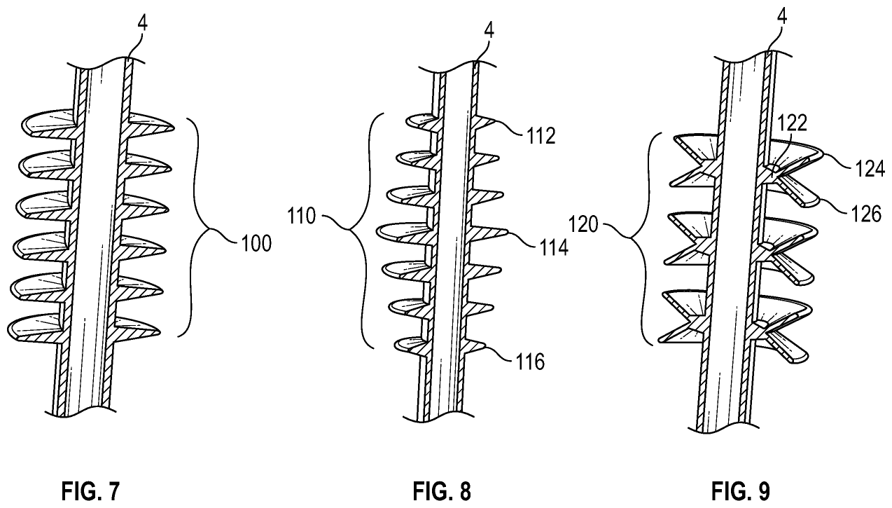

FIG. 7 is a cross-sectional view of an embodiment having tapered baffles 100 that are thicker adjacent to the tubular portion 4 and gradually become thinner moving radially outward, providing a flexible edge and that is non-traumatic to the tracheal mucosa. The baffles 100 are equal in diameter. FIG. 8 is a cross-sectional view of an embodiment having tapered baffles 110 that vary in diameter. The baffles 112 and 116 toward the proximal and distal ends have smaller diameters while the middle baffles 114 have larger diameters. Such varying diameter baffles can provide a better fit where the trachea has a non-constant inner diameter, such as a bulge or narrowing.

FIG. 9 is a cross-sectional view of an embodiment having baffles 120 that each include both a proximally angled portion 124 and a distally angled portion 126 extending from an inner base portion 122. Having baffle portions angled in both directions can provide better sealing and migration resistance in both directions.

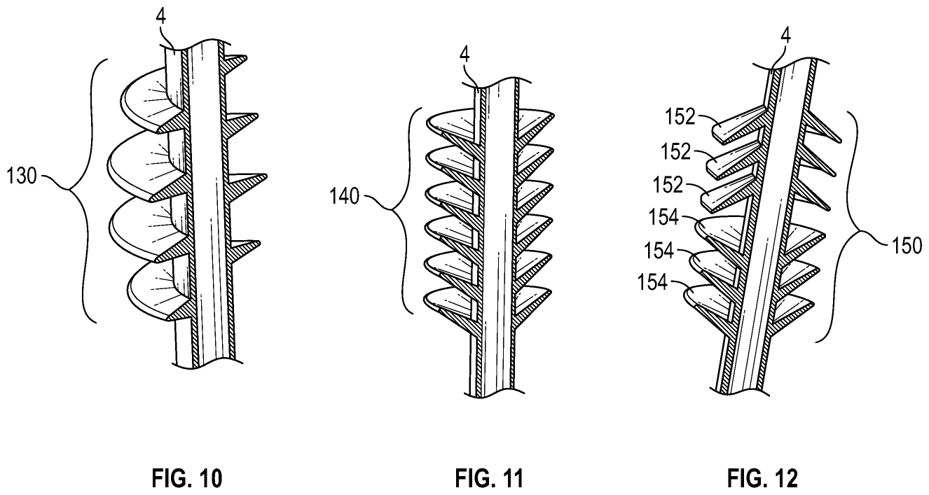

FIG. 10 is a cross-sectional view of an embodiment having a tapered, helical baffle 130 of varying radius, with a larger radius in the middle and smaller radii at the ends. FIG. 11 is a cross-sectional view of an embodiment having tapered, proximally angled baffles 140.

FIG. 12 is a cross-sectional view of an embodiment having tapered baffles 150 comprising one set of distally angled baffles 152 and one set of distally angled baffles 154, with the two sets tilted toward each other. Having baffles angled in both directions can provide better sealing and migration resistance in both directions.

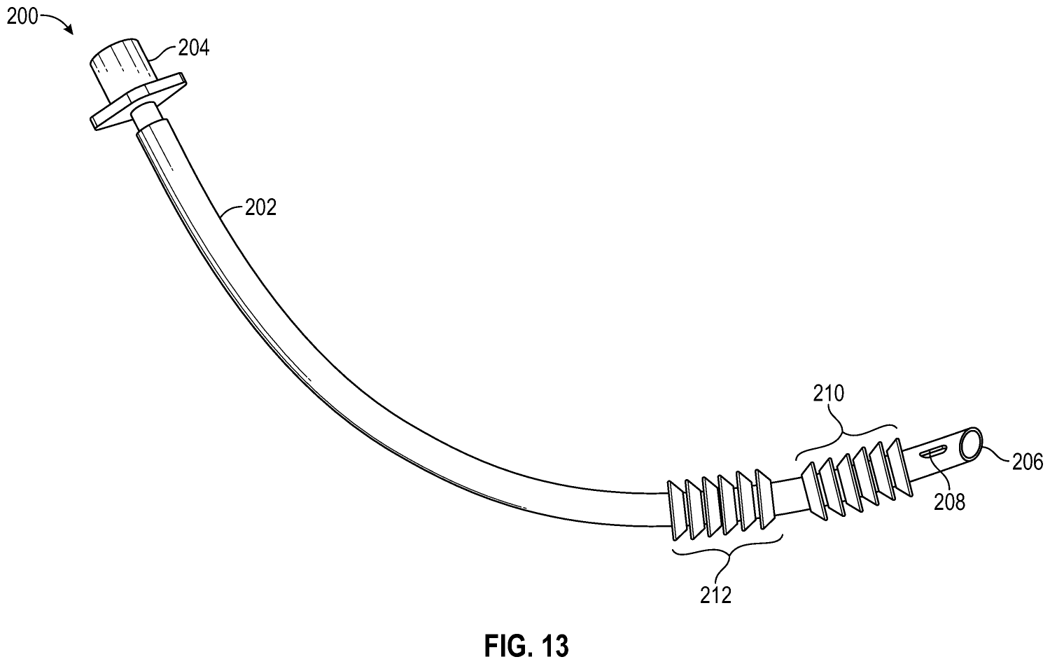

FIG. 13 shows an exemplary endotracheal tube 200 comprising a tubular portion 202, proximal adaptor 204, distal opening 206, lateral opening 208, a set of distally angled baffles 210, and a set of proximally angled baffles 212, with the two sets tilted away from each other. The device 200 is illustrated with a gap between the two sets 210, 212, which can vary in length from zero to several centimeters. Similarly, the number of baffles in each set can vary from one to six, or more. Analogously, any of the embodiments disclosed herein can have any number of baffles in each set of baffles, and those with multiple sets of baffles can have any spacing between the sets. Furthermore, the diameter of the baffles in any embodiment can vary from just greater than the diameter of the tubular portion (e.g., small annular ribs) to several centimeters greater than the diameter of the tubular portion.

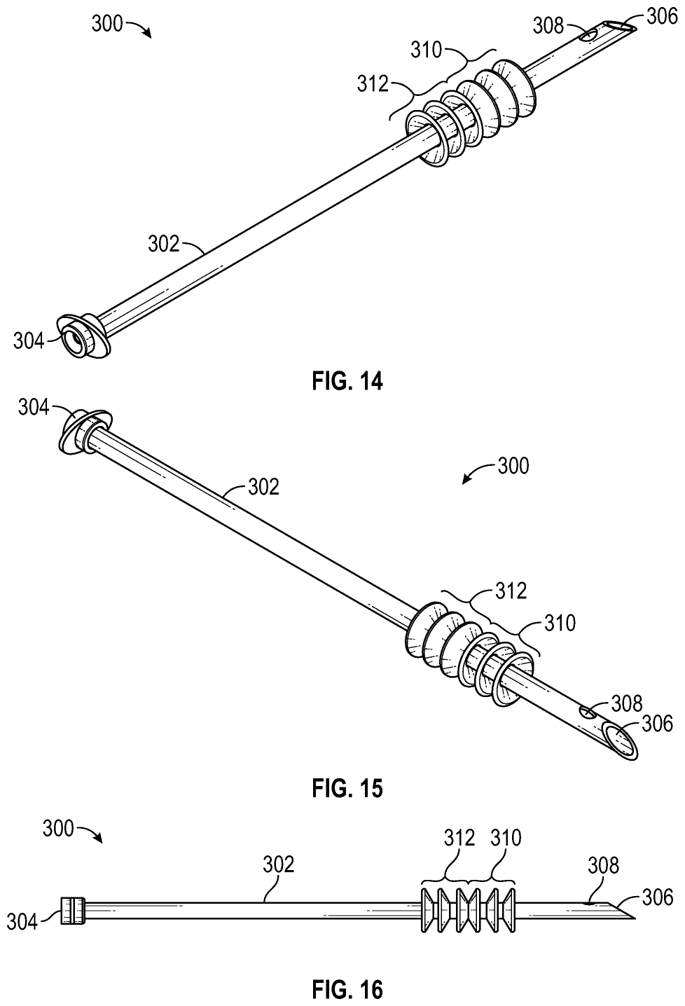

FIGS. 14-16 show views of another exemplary device 300 having a tubular portion 302, a proximal adaptor 304, a distal opening 306, a lateral opening 308, a set of distally angled baffles 310, and a set of proximally angled baffles 312, with the two sets positioned close together and tilted away from each other.

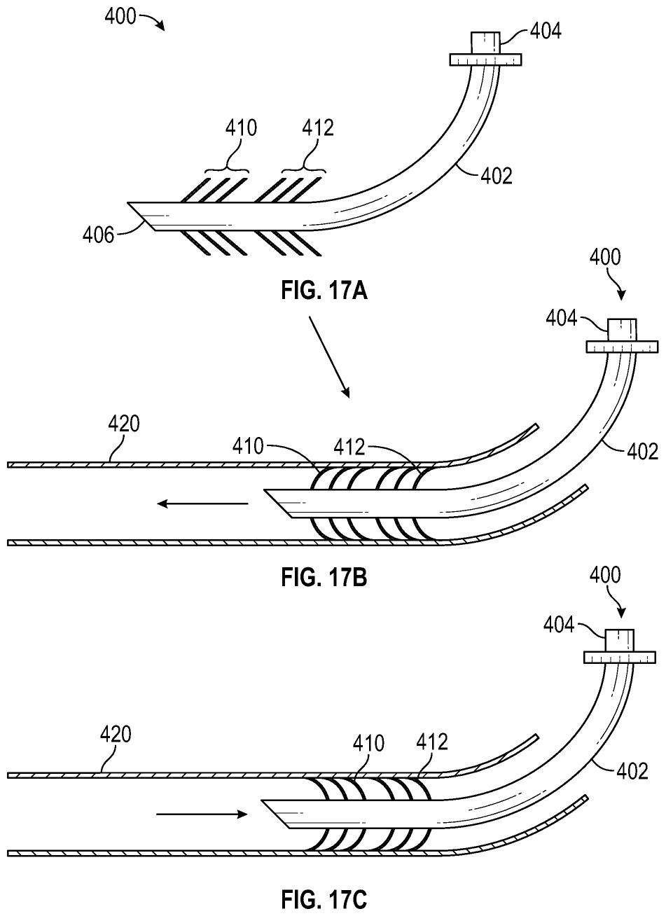

FIG. 17A schematically illustrates an exemplary endotracheal tube 400 having a tubular portion 402, proximal adaptor 404, distal opening 406, and two sets of proximally angled annular baffles 410 and 412 (each similar to the baffles 60). FIG. 17B illustrates how inserting the device 400 into a trachea 420 causes the baffles 410 and 412 to defect proximally as they contact the inner surface of the trachea. The diameter of the baffles is larger than the inner diameter of the trachea, forcing the flexible baffles to deform further proximally as the device 400 moves distally. Conversely, FIG. 17C illustrates how retracting the device 400 proximally relative to the trachea 420 causes the baffles to defect the other direction, distally, due to their engagement with the trachea. This phenomenon can be employed beneficially when placing any of the devices disclosed herein in a patient, as small adjustments of the device either proximally or distally within the trachea can re-orient the baffles in different directions, allowing a user to select whichever baffle orientation is more desirable for a given patient situation, and re-orient the baffles at any time without having to remove the device from the trachea.

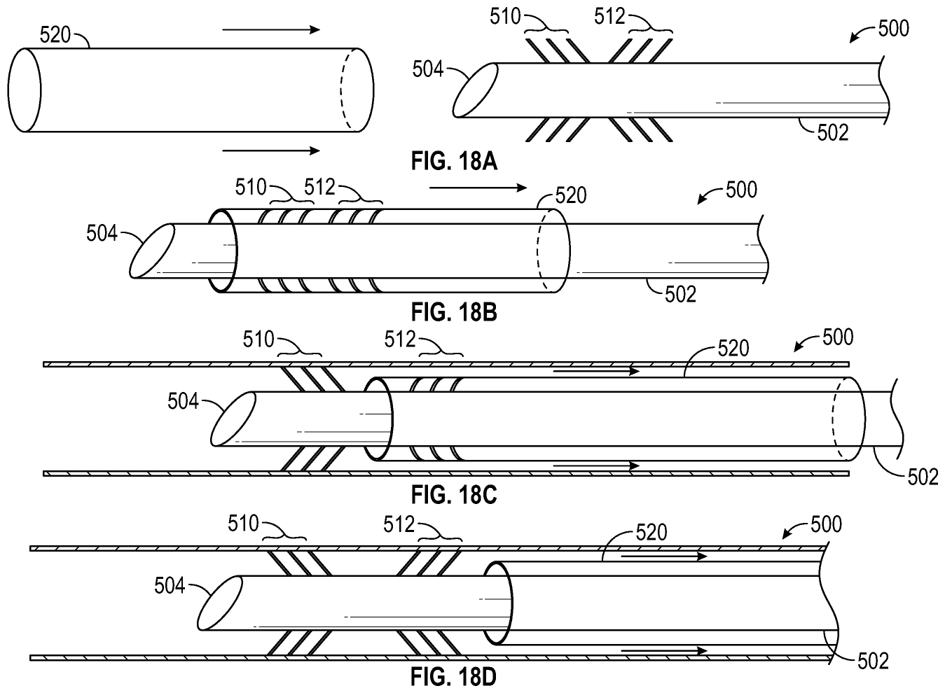

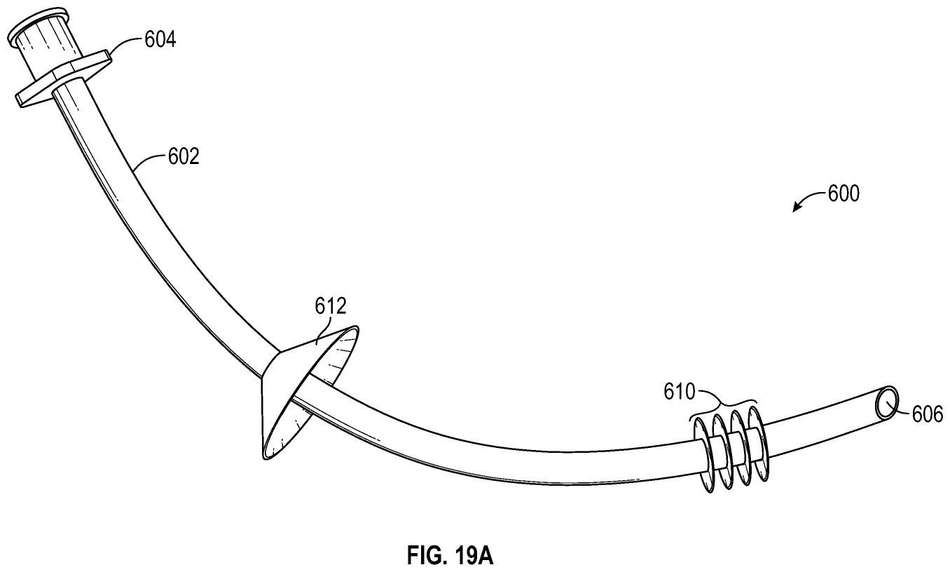

FIGS. 18A-18D illustrate how an additional loading tube can be used with any of the endotracheal tubes disclosed herein to better control the orientation of the baffles within the trachea. Without using such a loading tube, all of the baffles may end up angled in the same direction within the trachea. FIG. 18A shows an exemplary endotracheal tube 500 having a tubular portion 502, a distal opening 504, a distal set of distally angled baffles 510, and a proximal set of proximally angled baffles 512, along with a loading tube 520. The loading tube 520 can be passed over the baffles prior to insertion of the device into the patient, as shown in FIG. 18A. The device 500 with the loading tube 520 covering the baffles can then be inserted into to the trachea. As shown in FIG. 18C, the loading tube 520 can then be partially retracted proximally relative to the device 500 and the trachea, uncovering the distal set of baffles 510 and allowing them to resiliently unfurl into contact with the trachea. In order to then ensure the baffles 510 are angled distally (as shown in FIG. 18C), the device 500 and loading tube 520 can be moved proximally together relative to the trachea a small distance. Then, with the distal baffles 510 desirably oriented, the loading tube 520 can be moved further proximally while the device 500 is held steady relative to the trachea (as shown in FIG. 18D) to release the proximal baffles from the loading tube. Using this method, the device 500 (or other multi-baffle embodiment disclosed herein) can be deployed in the trachea with one set of baffles angled distally and another set of baffles angled proximally.

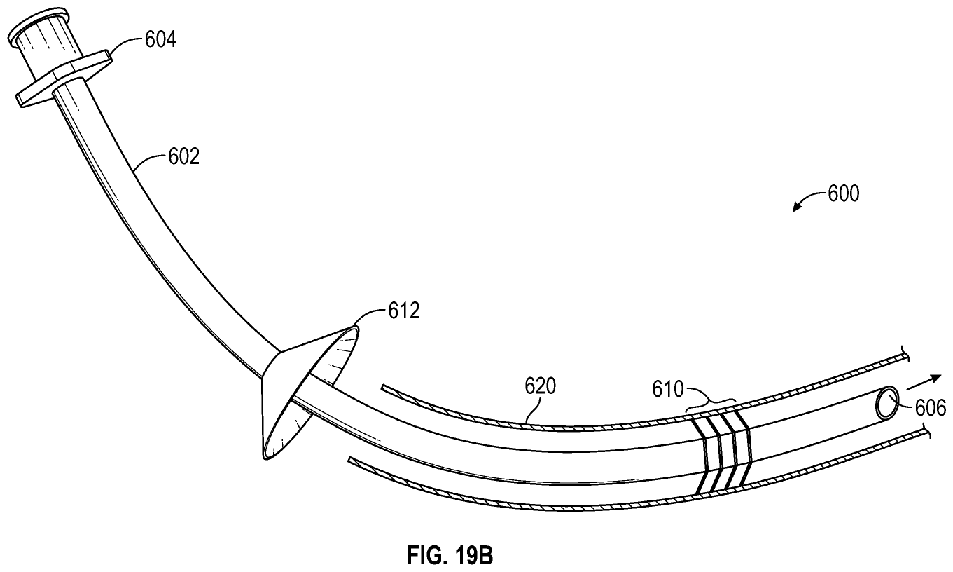

FIG. 19A shows an exemplary endotracheal tube 600 comprising a tubular portion 602, proximal adaptor 604, distal opening 606, a set of baffles 610, and a supraglottic umbrella 612 positioned around the tubular portion proximal to the baffles. The umbrella 612 can be positioned over the supraglottic larynx to protect the vocal cords and subglottic space from aspiration of gastric contents or upper airway fluids, and can redirect such material safely into the esophagus. In some embodiments, a proximal umbrella clears blood and other secretions from the hypopharynx/upper airway with removal. FIG. 19B shows the device 600 of FIG. 19A inserted into the trachea 620 with the supraglottic umbrella 612 positioned near the proximal end of the trachea, where the glottis would be. The umbrella can also function to divert other tubes, devices, and secretions toward the esophagus, promoting passage into the esophagus and preventing unintended passage into the larynx and trachea with potential trauma.

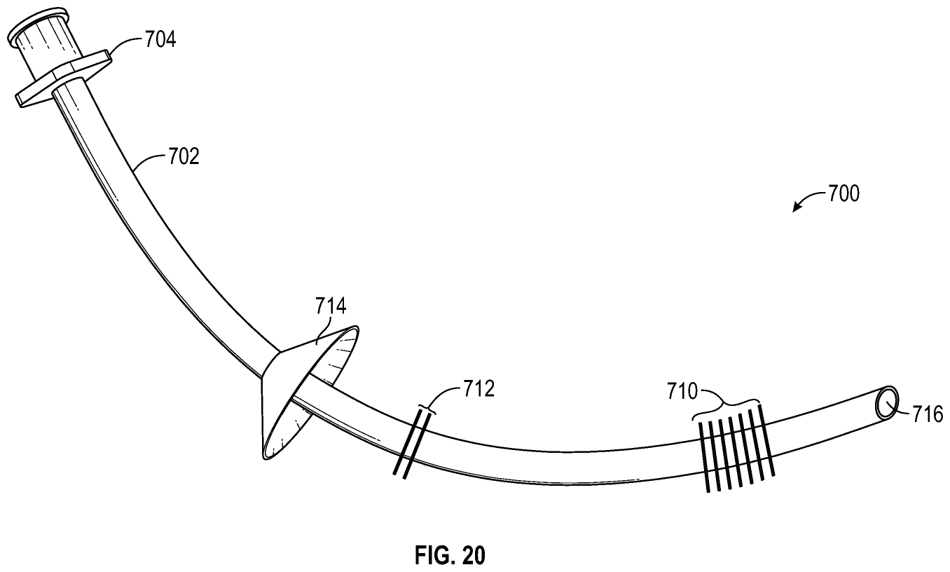

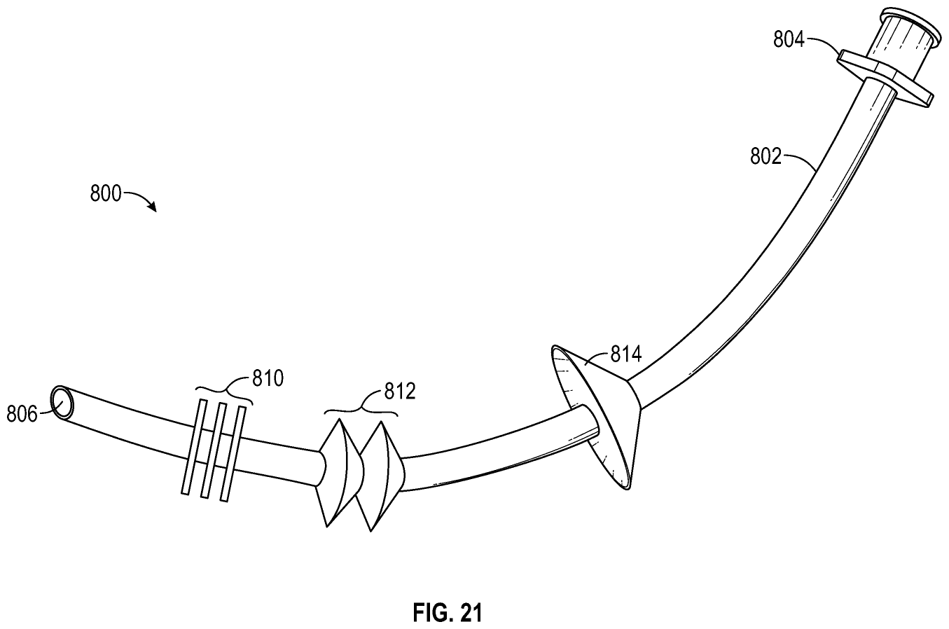

Similarly, FIG. 20 shows an exemplary endotracheal tube 700 comprising a tubular portion 702, a proximal adaptor 704, a distal opening 716, a distal set of baffles 710, a proximal set of baffles 712, and a supraglottic umbrella 714. The second set of baffles 712 can provide enhanced sealing, anchorage, and paratubular blockage.

FIG. 21 shows an exemplary endotracheal tube 800 comprising a tubular portion 802, a proximal adaptor 804, a distal opening 806, a distal set of baffles 810, a supraglottic umbrella 814, and an accordion-like expandable baffle portion 812 positioned between the distal baffles 810 and the umbrella 814. The expandable baffle portion 812 can allow for adjustment in the axial distance between the distal opening 806, the baffles 810, and the umbrella 814, to accommodate different sized anatomies and changes in tracheal dimensions with the respiratory cycle and head/neck movement. The expandable baffle portion can be about the same diameter as the other baffles, or can be smaller or larger. In some embodiments, an expandable baffle portion can be positioned elsewhere along the tubular portion, such as proximal to the supraglottic umbrella or between two sets of baffles. In some embodiments, two or more of such expandable baffle portions can be included. The extendibility of the tubular portion can help accommodate changes in shape and can help prevent excessive pressure on the back of the throat that can result in mucosal erosions. Such extendable baffle portions can provide any range of extensibility, such as from a few millimeters to several centimeters (e.g., 1-2 centimeters).

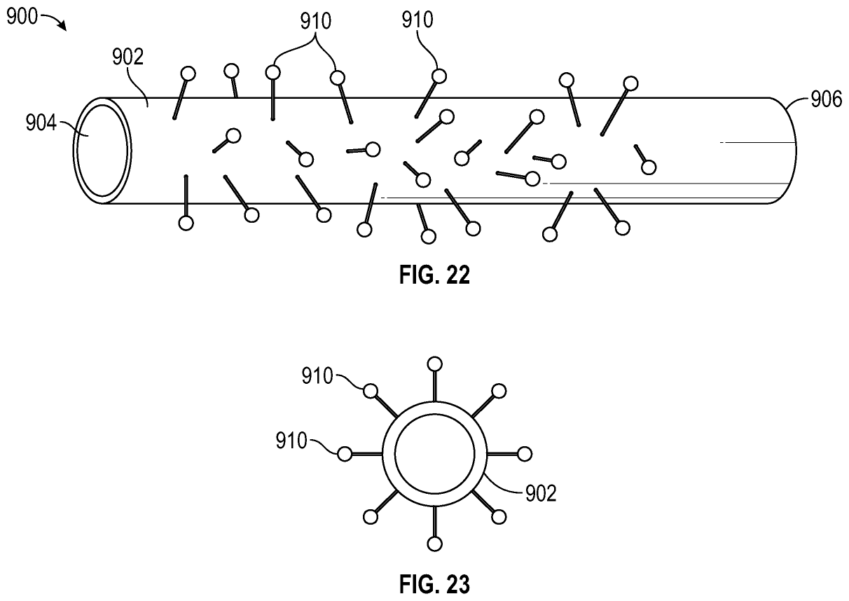

FIG. 22 shows an exemplary endotracheal tube 900 comprising a tubular portion 906, a distal opening 904, and a plurality of discrete, flexible protuberances or projections 910 extending radially from along the outer surface of the tube. FIG. 23 shows an end view of the tube 906 with an exemplary evenly spaced apart projection orientation. In other embodiments, the protuberances 910 can be more randomly spaced and randomly oriented along the tube 906 to provide more even contact with the trachea and fewer and more tortuous flow pathways between the protuberances. Like baffles, the protuberances 910 can provide sealing while also limiting migration. Such protuberances can be combined with baffles in some embodiments as well. Any of the baffle embodiments disclosed herein can be optionally enhanced with added protuberances along the tubular portion adjacent to the baffle (e.g., proximal to, distal to, or between the baffles).

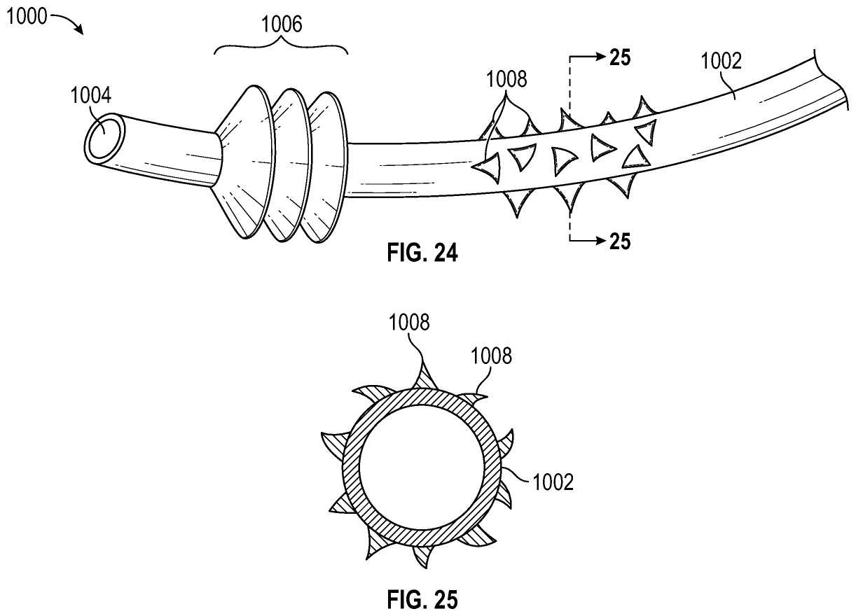

FIG. 24 shows an exemplary endotracheal tube 1000 comprising a tubular portion 1002, a distal opening 1004, a set of annular baffles 1006, and a plurality of discrete, flexible protuberances 1008 extending radially from along the outer surface of the tube. FIG. 25 shows an end view of the tube 1002 with an exemplary orientation of protuberances 1008. The protuberances 1008 can have a curled or tilted terminus to aid in contacting the trachea. Like baffles 1006, the protuberances 1008 can provide sealing while also limiting migration. Any of the baffled embodiments disclosed herein can be optionally enhanced with added protuberances similar to protuberances 1008 along the tubular portion adjacent to the baffles (e.g., proximal to, distal to, or between the baffles).

In other embodiments, the baffles can comprise eccentric and/or non-continuous members that include circumferential spaces or gaps, which can help the baffles deform more readily and allow them to better conform to the shape of the trachea. For example, in some embodiments the baffles can be oval, egg-shaped, or other non-circular rounded shapes. In some embodiments, non-continuous baffle members can comprise one or more circumferential gaps, plural adjacent lobes, or an annular or semi-annular ring of protuberances or projections extending radially from the tubular portion.

In some embodiments, the baffles can help resist axial movement of the endotracheal tube from high ventilation pressures. In some embodiments, baffles angled proximally allow escape of high ventilation pressures and thereby reduce the risk of unwanted proximal migration of the tube.

In some embodiments, the tube prevents aspiration due to cuff deflation during performance of tracheostomy. In some embodiments, the baffles adjust their contour to maintain a seal with inspiration and expiration and accommodate expansion and contraction of the airway during the respiratory cycle. In some embodiments, the baffles can have a curled perimeter edge, which can help maintain orientation (prevents flipping of direction) with proximal and distal movement of the tube. In some embodiments, the multiple baffles create closed compartments, which can limit biofilm formation on the distal part of the tube. In some embodiments, the multiple baffles create closed compartments between the baffles that prevent air escape, limit oxygen desaturation, and lessen the risk of airway fire during tracheostomy procedure. In some embodiments, the baffles minimize compression of the esophagus along the posterior tracheal wall, thereby avoiding dysphagia associated with inflated balloon cuffs. Some embodiments minimize effects on swallowing through spacing of baffles or specially shaped baffles. In some embodiments, the endotracheal tube with baffles has multiple projections or protuberances that allow torsion and flexibility of the device.

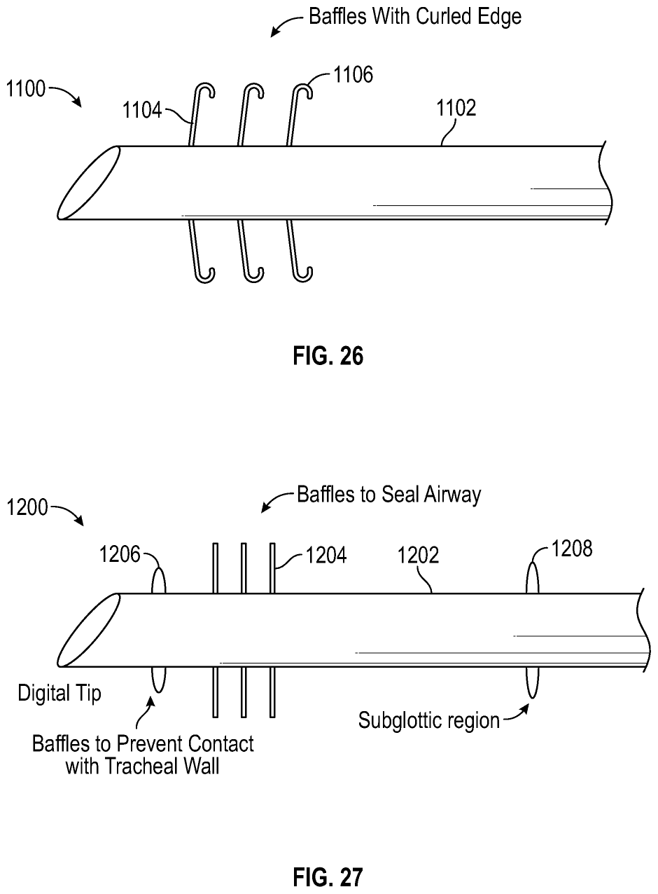

FIG. 26 shows an exemplary endotracheal tube 1100 that comprises a set of baffles 1104 that have curled edges. In this example, the edges are curls proximally, while in other embodiments the edges of the baffles can be curled distally in a similar manner. The degree of the curling can vary. In some embodiments, the edges can curl around 90 degrees such that the edges point proximally. In other embodiments, the edges can curl around 180 degrees such that the edges point radially inwardly (similar to as shown in FIG. 26). In other embodiments, the edges of the baffles can be curled more than 180 degrees. In some embodiments, the entire annular peripheral edge of the baffle is curled, while in others only parts of the peripheral edge are curled.

FIG. 27 shows an exemplary endotracheal tube 1200 having a set of broader baffles 1204 to seal the airway, along with narrower proximal and distal baffle to prevent contact with the tracheal wall. A narrower distal baffle 1206 is positioned distal to the baffles 1204 nearer to the distal tip of the tube 1200, and a narrower proximal baffle 1208 is positioned proximal to the baffles 1204 at the subglottic region. The narrower baffles help prevent the tube from contacting the tracheal wall, such as when the tube flexes or bends to conform to the native anatomy.

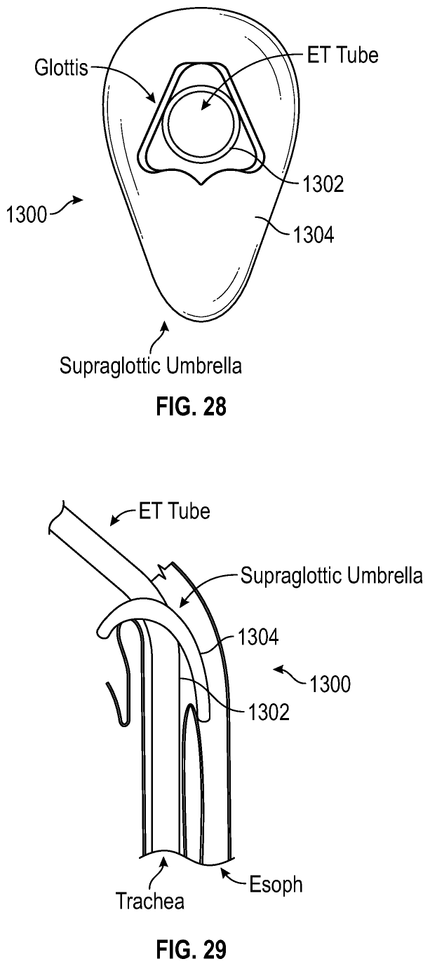

FIGS. 28 and 29 show an endotracheal tube 1300 having tubular portion 1302 and a supraglottic umbrella 1304 positioned over the glottis. The umbrella 1304 extends radially from the tubular portion 1302, with the posterior aspect extending further than the rest of the umbrella to extend toward and/or into the esophagus. As shown in the cross-sectional side view of FIG. 29, the anterior aspect of the umbrella can extend up and over and/or in front of the glottis, while the elongated posterior aspect can extend back and downwardly into the esophagus, creating a barrier to prevent materials (e.g., from the mouth or esophagus) from inadvertently entering the trachea while the tube is in place, and visa versa. The umbrella 1304 can also help standardize the distance of the tube's distal end from the glottis.

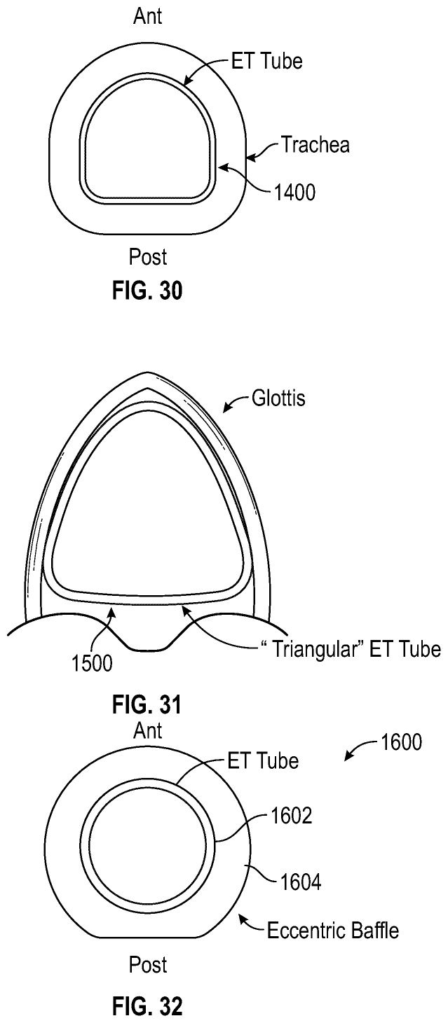

FIG. 30 shows an exemplary non-circular cross-sectional profile 1400 for an endotracheal tube, with the posterior aspect having a flattened shape. This non-circular profile for a tube can better match the native shape of the trachea/glottis. The anterior aspect is curved, while the posterior aspect is straight, or at least less curved than the anterior aspect. The lateral aspects of the profile can be curved or straight, or have an intermediate curvature that transitions from the curved anterior aspect to the flattened posterior aspect. The posterior-lateral aspects of the profile can comprise corners or more sharply curved aspects.

FIG. 31 shows a generally triangular cross-sectional profile 1500 for an endotracheal tube that is similar to the profile 1400, but where the profile has a more triangular shape with three flattened sides or straighter aspects (the posterior aspect and two anterior-lateral aspects), and three corners or more sharply curved transitions between the three flattened sides, forming a generally triangular shape. This shape can better match the geometry of the glottis.

In other embodiments, an endotracheal tube can have an elliptical cross-sectional profile with a greater diameter in the anterior-posterior direction than in the lateral direction, which can better accommodate the native shape of the trachea, such as in the sub-glottic region (e.g., cricoid ring).

FIG. 32 is a cross-sectional view of an exemplary endotracheal tube 1600 having a tubular portion 1602 and non-circular or eccentric baffle 1604 that has a flat posterior aspect. In other embodiments, eccentric or non-circular baffles can include two, three, or more flat or flattened aspects. In some embodiments, an endotracheal tube can include elliptical baffles, pear-shaped or egg-shaped baffles, and/or other non-circular shapes. Any of the baffle configurations disclosed herein may be oriented perpendicular to the tube wall or non-perpendicular, such as projecting at an acute angle relative to the tube wall.

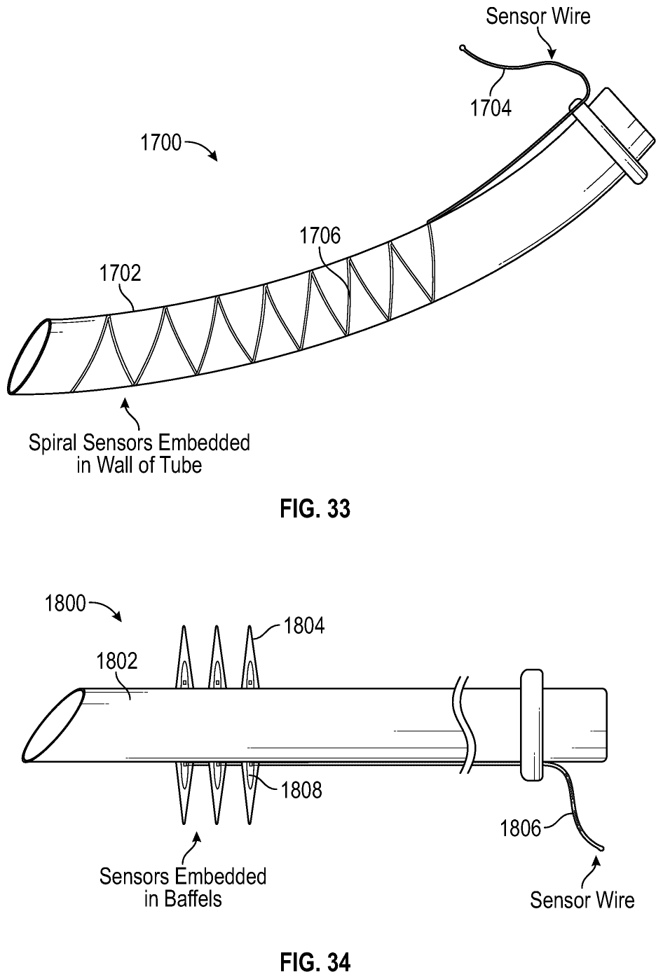

FIG. 33 shows an exemplary endotracheal tube 1700 that includes a tubular wall 1702, a sensor wire 1704, and a helical or spiral sensor 1706 embedded in the wall of the tubular portion and coupled to the wire. The senor 1706 can detect the presence of another object, such as an adjacent feeding tube, and/or can measure pressure, contact, position, deflection, movement, or other properties. The tube 1700 can also include one or more baffles in addition to the sensor 1706. The helical sensor(s) 1706 can also be used with integrated vocal cord nerve monitoring electrodes in some embodiments.

FIG. 34 shows an exemplary endotracheal tube 1800 that includes a tubular portion 1802, a set of baffles 1804, a sensor wire 1800, and sensors 1808 embedded in the baffles and coupled to the wire. The sensors 1808 can be annular, or partially annular, or can be present in a discrete array dispersed around the baffle, or otherwise arranged within the baffle. Such sensors can detect the presence of another object, such as an adjacent feeding tube, and/or can measure pressure, contact, position, deflection, movement, or other properties.

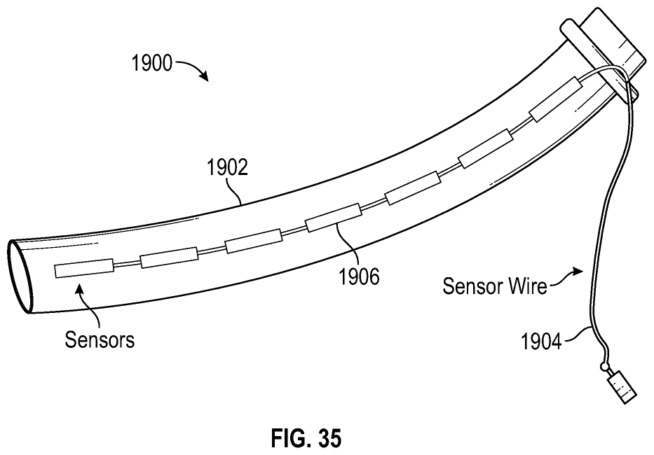

FIG. 35 shows an exemplary endotracheal tube 1900 that includes a tubular portion 1902, a sensor wire 1904, and a series of axially spaced apart sensors 1906 along the tube and coupled to the wire. Such sensors can detect the presence of another object, such as an adjacent feeding tube, and/or can measure pressure, contact, position, deflection, movement, or other properties. The tube 1900 can also include one or more baffles in addition to the sensor 1906.

In addition to the features disclosed elsewhere here, the following features can also be included in any baffled tube embodiment. In some embodiments, the lumen has a triangular cross-sectional shape at the level of the glottis to minimize traumatic injury to the glottic larynx. In some embodiments, elliptical baffles are angled to facilitate insertion through the glottis by entering the posterior glottis first and displacing the vocal cords from posterior to anterior as the baffle passes. In some embodiments, spacing of baffles prevents mucosal injury to subglottic mucosa associated with prolonged intubation. In some embodiments, the shape of the baffles will prevent accidental passage of nasogastric or orogastric feeding tubes into the airway. In some embodiments, a supraglottic umbrella anchors the tube superiorly, assuring that the tube is fully inserted and preventing contact of tip with carina or distal insertion into the mainstem bronchus (especially in women). In some embodiments, the position of the supraglottic umbrella can be adjustable to vary the length of the tube distal to the glottis. In some embodiments, an elliptical shape fits the contour of the subglottic region better, thereby providing a maximal airway without contact with subglottic mucosa. In some embodiments, a spiral baffle design allows greater contact of nerve monitoring electrodes with vocal cords. In some embodiments, a triangular lumen may maintain orientation of tube within the trachea and minimize trauma to glottis. In some embodiments, a proximal set of baffles anchors the tube at the level of the glottis and prevents pooling of secretions in the subglottic airway. In some embodiments, the design of the baffles alters the maximal ventilation pressure at which leakage of air occurs. In some embodiments, variations in shape, thickness, and stiffness minimize pressure exerted on the esophagus posteriorly to limit dysphagia associated with cuffed tubes. In some embodiments, sensors along the length of the tube will identify contact of the tube with the airway at multiple points and measure amount of pressure. In some embodiments, sensors will detect glottic movement/changes in pressure. In some embodiments, sensors will detect deflection or orientation of baffles and movement of the tube. In some embodiments, sensors will detect directional movement of the tube. In some embodiments, sensors will detect shape or deformation of tube. In some embodiments, proximal glottic baffles at varying distance from the distal baffles allow accurate placement of tube at the optimal distance from the carina and subglottic region.

Benefits of the disclosed baffled tubes can include any of the following: (1) prevention of aspiration and ventilator-associated tracheobronchitis/pneumonia; (2) prevention of distal and proximal tube migration; (3) avoidance of risks of pressure necrosis from high cuff pressures; (4) prevention of tracheal stenosis; (5) avoidance of complications from continuous subglottic suction; (6) prevention of aspiration of gastric contents; (7) prevention of tube migration due to airway movement associated with the respiratory cycle or changes in head/neck position; (8) safer tracheostomy procedure; and/or any other benefits or advantages disclosed elsewhere herein, in any combination.

### Tracheostomy Tubes with Baffles

Baffled tube designs analogous to those described elsewhere herein can also be used for tracheostomy tubes, with similar features and advantages. Exemplary benefits of a tracheostomy tube with baffles include any one or more of the following: (1) easier insertion and removal; (2) prevention of aspiration and ventilator-associated tracheobronchitis/pneumonia; (3) prevention of distal and proximal tube migration; (4) avoid risks of pressure necrosis from high cuff pressures; and (5) avoid rupture of cuff during insertion and cuff malfunction.

In addition to the other benefits described herein, other benefits can be provided by tracheostomy tubes with baffles. In some tracheostomy tubes with baffles, internal and external baffles at the stoma prevent accidental decannulation. In some tracheostomy tubes with baffles, a spiral baffle allows phonation. In some tracheostomy tubes with baffles, baffles center the tube within the tracheal lumen and prevent impingement on the tracheal wall.

### Esophageal Stents with Baffles

Baffled tube designs analogous to those described elsewhere herein can also be used for esophageal stents, with similar features and advantages. For example, esophageal stents with baffles can help to prevent migration of the stent. In some embodiments, esophageal stents with baffles can comprise a continuous helical or spiral baffle. Compared to embodiments with discrete separated baffles, a continuous spiral baffle can allow secretions and other materials to pass without stasis and can avoid focal circumferential pressure on the esophagus. Some esophageal stent embodiments can include multiple independent protuberances, projections, stalks, or bristles that anchor the tube but allow greater flexibility (in addition to baffles, or instead of baffles). Exemplary benefits of esophageal stents with baffles can include: (1) prevention of proximal and distal migration; (2) avoid complications associated with suturing of stent; and (3) more effective sealing of luminal perforation.

### General Considerations

Characteristics, materials, and other features described in conjunction with a particular aspect, embodiment, or example of the disclosed technology are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means physically coupled or linked and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the full scope of the following claims. We therefore claim all that comes within the scope of these claims and their equivalents.

1. An endotracheal tube comprising:
a tubular portion;
a proximal adaptor at a proximal end of the tubular portion;
a distal opening at or adjacent to a distal end of the tubular portion; and
a set of baffles positioned around the tubular portion between the proximal adaptor and the distal opening;
wherein the baffles are configured to contact an inner wall of a trachea when the endotracheal tube is inserted into the trachea, such that the baffles resist migration of the endotracheal tube relative to the trachea and block aspiration between the tubular portion and the inner wall of the trachea.

2. The endotracheal tube of claim 1, wherein the baffles are fully annular.

3. The endotracheal tube of claim 1, wherein the baffles are angled proximally from the tubular portion.

4. The endotracheal tube of claim 1, wherein the baffles are angled distally from the tubular portion.

5. The endotracheal tube of claim 1, wherein the baffles extend perpendicularly from the tubular portion.

6. The endotracheal tube of claim 1, wherein the baffles comprise a material that is resiliently deformable and conforms to contours of the trachea.

7. The endotracheal tube of claim 1, wherein the endotracheal tube does not include an inflatable cuff.

8. The endotracheal tube of claim 1, wherein the set of baffles comprises two or more distinct groups of baffles.

9. The endotracheal tube of claim 1, wherein the set of baffles comprises a first group having baffles that are angled proximally from the tubular portion and a second group having baffles that are angled distally from the tubular portion.

10. The endotracheal tube of claim 1, wherein the baffles comprise a helical baffle.

11. The endotracheal tube of claim 1, wherein the set of baffles comprises individual baffles that each has a base portion extending from the tubular portion, a first angled portion extending proximally from the base portion, and a second angle portion extending distally from the base portion.

12. The endotracheal tube of claim 1, wherein the baffles are tapered from a greater thickness adjacent to the tubular portion to a lesser thickness farther radially from the tubular portion.

13. The endotracheal tube of claim 1, wherein the baffles have different diameters.

14. The endotracheal tube of claim 1, further comprising a loading tube that is positioned around the set of baffles before the baffles are inserted into the trachea and is configured to be retracted off of the baffles after the baffles are positioned within the trachea.

15. The endotracheal tube of claim 1, further comprising a supraglottic umbrella positioned around the tubular portion proximal to the set of baffles, the umbrella configured to block aspiration of materials into the trachea.

16. The endotracheal tube of claim 1, further comprising an axially expandable baffle portion positioned proximal to the set of baffles, the expandable baffle portion having a greater diameter than the tubular portion.

17. The endotracheal tube of claim 1, further comprising a plurality of flexible protuberances extending from the tubular portion.

18. The endotracheal tube of claim 1, wherein the baffles comprise a first material and the tubular portion comprises a second material that is different than the first material, and wherein the baffles are formed separately from the tubular portion.

19. The endotracheal tube of claim 1, wherein the tubular portion or the baffles comprises reinforcing elements in selected regions to provide asymmetric reinforcement.

20. The endotracheal tube of claim 1, wherein a distal end portion of the tubular portion forks into two forks, each fork having baffles.

21. The endotracheal tube of claim 1, further comprising at least one suction channel extending along the tubular portion and fluidly coupled to at least one opening adjacent to the baffles.

22. The endotracheal tube of claim 1, wherein the tubular portion has a non-circular cross-sectional profile.

23. The endotracheal tube of claim 22, wherein the tubular portion has a triangular cross-sectional profile.

24. The endotracheal tube of claim 22, wherein the tubular portion has a flattened posterior aspect.

25. The endotracheal tube of claim 22, wherein the tubular portion has an elliptical cross-sectional profile.

26. The endotracheal tube of claim 1, wherein at least one of the baffles has a flattened posterior aspect.

27. The endotracheal tube of claim 1, wherein at least one of the baffles has a curled perimeter edge.

28. The endotracheal tube of claim 1, further comprising a supraglottic umbrella that has a non-circular shape.

29. The endotracheal tube of claim 28, wherein the umbrella has a posterior portion that extends to a greater radial distance from the tubular portion than an anterior portion, such that the posterior portion can extend over or into the esophagus.

30. The endotracheal tube of claim 1, further comprising a sensor wire and at least one sensor embedded in the tubular portion or the baffles.

31. The endotracheal tube of claim 30, wherein the at least one sensor comprises a helical sensor embedded in the tubular portion.

32. The endotracheal tube of claim 30, wherein the at least one sensor comprises sensors embedded in the baffles.

33. The endotracheal tube of claim 30, wherein the at least one sensor comprises a series of sensors axially spaced apart along the tubular portion.

34. A tracheostomy tube comprising the features of claim 1.

35. An esophageal stent comprising the features of claim 1.

36. A tubular device for placement in any anatomical tubular structure or orifice comprising the features of claim 1.
